(19) United States
(12) Patent Application Publication
(10) Pub. No.: US 2003/0088158 A1
(43) Pub. Date: May 8, 2003

(54) Expander for the oral cavity's rehabilitation

(76) Inventor: Kuo-Feng Chien, Yun Lin Hsien (TW)

Correspondence Address: Alan D. Kamrath, Rider, Bennett, Egan & Arundel, LLP, Suite 2000, 333 South Seventh Street, Minneapolis, MN, 55402, US

(21) Appl. No.: 10/012,717

(22) Filed: Nov. 5, 2001

(51) Int. Cl. A61B 1/32

(52) U.S. Cl. 600/237

(57) ABSTRACT

The present invention relates to an expander for the oral cavity's rehabilitation ' and more particularly to a kind of elastic clip having one adjusting end and one occlusive end thereat, wherein a screw is pivoted onto the upper brace of the said adjusting end, and then one end of the screw is pushed through a lower brace, while a nut is screwed onto the said screw at the portion in-between the upper and lower braces, or alternatively, having a spring and a nut be mounted successively onto the said screw after it has been pushed through the said lower brace, and therefore it would be applicable to do the necessary exercise with different extent in opening her or his mouth and muscular force while an user is adjusting both the opening distance and also the elastic tension of the occlusive end positioned at the other end of the said elastic clip, by means of adjusting each nut, and thus while a patient is biting at the said occlusive end of the said elastic clip, the patient would be able to achieve with a proper occlusive movement and exercise by simply utilizing the elastic tension caused by the said occlusive end of the said elastic clip accordingly.

### BACKGROUND OF THE INVENTION

1. Field of the Invention

In accordance with the present invention, a screw, which is pushed through a lower brace, is pivoted onto a upper brace positioned at the adjusting end of an elastic clip, more over a nut, which is used for adjusting the occlusive end of the said elastic clip, is then screwed at a position in-between the upper and lower braces, in order for a user to do the necessary exercise with different extent in opening her or his mouth by means of adjusting the said occlusive end thereat, and thus while a patient is biting at the said occlusive end of the said elastic clip, the patient would be able to achieve with a proper auxiliary movement and exercise by simply utilizing the function of elastic tension caused by the said elastic clip accordingly.

2. Background of the Invention

The oral cancer is some kinds of malignant tumors infected in oral cavity as well as related tissues of the oral cavity, however the chronic incitements, such as chewing betelnut, smoking, drinking, syphilis, over-sunburned, bad oral hygiene, residual root, and also darning caused by improper root planning skills, are one the Infectious agents for the oral cancers, among that the particular cause for oral cancer which relates to the chewing betelnut has become the main cause for infecting oral cancer in spite that the oral cancer has leapt to the seventh position of the top ten cancers infected in Taiwan area; the most patients of oral cancer are accepting the formal medical treatment only at the later period of her or his disease, and in order to cure their illnesses and save their lives, the general treatments include surgical operation, or in conjunction of the Radiation Therapy, the Chemotherapy, and it is needed for carrying out a extensive surgical resection in order to cure the tumors, which sometimes having a preventive or Therapeutical cervical lymphatolysis in advance, as a large size of tumor would result in infirmity and lacuna on both oral cavity and facial tissues, and further the problems such as the difficulty in masticatory function, going through in-between the mouth and nose organs; Besides the problems caused by the Radiation Therapy including Stomatitis, difficulty in opening/closing mouth, Ulcers, and inflamed by the facial cauterization; in the meantime the daily self-reinforced yawning movement training is necessary to carry out for preventing form a lockjaw and tonic mandibular joints in conjunction with the oral cavity's rehabilitation program while after surgical resection on oral tissues, as it would cause problems of the fibrotic masticatory muscle, tonic, limitation of yawning movement etc., but the known exercise suggested by doctors for the yawning movement training, as illustrated in FIG. 1, is generally utilizing the bronchial scopes 2, which are piled up and then put into the position in-between the upper/lower large bicuspid teeth, in order to enlarging the opening extent for the oral cavity and preventing the situation of the lockjaw and tonic mandibular joints, a progressive multiplicative method (i.e. increasing the quantity of the bronchial scopes 2 progressively) is then usually taken.

Owing to the above said progressive multiplicative method with the bronchial scopes for doing the yawning movement training is a self-controlling type training method by the patient, therefore it would always being stopped by paining, and more over the enlarging movement on oral cavity forced by means of a plurality of the bronchial scopes is difficult to execute continuously and repeatedly, which is not only slow with the inferior result in training, but also it is very difficult in operating and also very easy to collapse while the bronchial scopes piled up too high.

### SUMMARY OF THE INVENTION

Thus, in order to overcome all above unpleasant defects occurred while doing exercise for yawning movement with bronchial scope, so it is desirable for the present invention to have a screw, which is pushed through a lower brace after it has been screwed on with a nut thereat, is pivoted onto the upper brace positioned at the adjusting end, which is under a constant closing condition, of an elastic clip therefrom; in addition there is a occlusive end, which is under a constant opening condition, configured at the other end of the said elastic clip, and in this way the opening extent of the said occlusive end could then be adjustable to meet the requirement on different extent for opening mouth of a user, hence by the time while a patient is biting at the said occlusive end of the said elastic clip, then she or he will be able to achieve a tense training program consecutively and repeatedly on her or his occlusive musculature just by means of the function of elastic tension of the said elastic clip, further to improve the effect of the relative exercises consequently.

It is, therefore, another objective of the present invention to have the above said screw installed on the said adjusting end to be mounted with a spring and also screwed on a nut after it had been pushed through the said lower brace, and in that way the spring will create different pressure on the said adjusting end by adjusting the said nut, and resulting in a further adjustment and proper controlling on the occlusive strength of the said occlusive end, thus the patient will then achieve a tense training on occlusive musculature with different occlusive strength accordingly.

It is a further object to this invention to provide a fixed soft padding onto the occlusive plane of the above said occlusive end, in order to enhance the frictional resistance and hence preventing the said occlusive end to be slipped off from the mouth and also having a further protection for teeth consequently.

### DETAILED DESCRIPTION OF THE PREFERRED EMBODIMENTS

Now referring to the FIGS. 2 and 3, the present invention comprises a elastic clip 1, wherein the said elastic clip 1 consisting of the upper and lower brace 11, 12, and further there are corresponding pivot lugs 111, 121, installed onto the corresponding portions in the center section of the said upper and lower brace 11, 12, and also a pivot pin 14 installed therefrom, in the meantime a torque helical spring 13 is not only coupled onto the said pivot pin 14 position in-between the said upper and lower brace 11, 12, but also having both ends of the said torque helical spring 13 to push against the bottom surfaces of the said upper and lower brace 11, 12 respectively, and in this way one end of the said elastic clip 1 is under a constant closing condition, and its other end is under a constant opening condition, in addition, the constant closing end of the said elastic clip is actually the adjusting end 1a, and the constant opening end of the said elastic clip is actually the occlusive end, wherein:

A pivot lug 112 is installed onto the said adjusting end 1a at the bottom surface in center portion of the said upper brace 11 therein, and more over a screw 17 is installed onto the upper flat portion 171, in such configuration the said upper flat portion 171 on the said screw 17 is then fixed onto the paired pivot lugs 112 by the said pivot pin 113, while a nut 15 and a washer 16 are screwed and coupled onto the said screw 17 successively, and then the other end of the said screw 17 is then pushed through the long boring 122 pierced into the center area of the lower brace 12.

As noted, there is a soft padding 124 is fixed outside of the said upper and lower brace 11, 12 respectively, therein the said soft padding 124 having a rising surface and also curve of the inner edge of the said rising surface is matching to the curve of the whole row of teeth.

Therefore, while screwing and adjusting the displacement of the said nut 15 screwed onto the screw 17 pivoted on the adjusting end 1a of the said elastic clip 1, in order to have a proper opening distance for the occlusive end 1b of the said elastic clip 1, then it would be applicable to have a desirable adjustment and controlling, such as the different opening performance of the said occlusive end 1b illustrating on the FIGS. 3 and 4, the purpose for such configuration is to allow a patient to freely adjust the said occlusive end according to different extent in opening mouth, that is, the opening distance of the occlusive end 1b of the said elastic clip a would be firstly set up at the smallest level, and then while the occlusive end 1b is put into a position in-between the upper and lower gums in the oral cavity and having both the upper/lower gums to bite at the soft padding 114, and in this way it is applicable to enhance the frictional resistance occurred among the upper/lower gums and the said occlusive end 1b and preventing the occlusive end to be slipped off from the mouth and also having a further protection for teeth consequently; afterwards if adjusting the nut 15 screwed onto the said screw 17 of the said occlusive end 1b, in order to enlarge the opening distance in-between the upper/lower braces 11, 12 of the said occlusive end 1b, then the mouth of the patient will be able to further open by a proper extent, continuously the patient could close her or his mouth thoroughly in order to close the opened occlusive end 1b, and while under the continuous and repeating exercises, it is not only applicable to prevent from a lockjaw but also could have a desirable tense training as well, which is very convenient to operate without any problems such as difficulty in piling it up and ease in collapse like a conventional bronchial scope.

In addition, with reference to FIGS. 5 and 6, a spring 18 and a nut 19 will be mounted with and screwed onto the above said screw 17′ position on the said occlusive end 1b after it had be pushed through the said lower brace 12, and in this way the different pressure would be created while the said spring 18 is forced against the said lower brace 12 by means of screwing and adjusting the displacement for the said nut 19, that is, except utilizing the relative elastic resistance created at the said occlusive end 1b of the said elastic clip 1, which is caused by the torque helical spring 13, the tense training for the occlusive strength on the said occlusive end 1b would be reinforced and achieve a desirable adjustment which is applicable to all patients if it is also utilizing the auxiliary function from the said spring 18 installed onto the said adjusting end 1a accordingly.

Next, referring to the FIG. 7, and as noted, while the said torque helical spring 13 which is mounted at the portion in-between the upper/lower braces 11, 12, is used for providing both the elastic and clipping functions for the said elastic clip 1, and alternatively if it is replaced by providing a compression spring 10 forced against at the end portion in-between the upper/lower braces 11, 12, then in this way it is still applicable to provide the said elastic clip 1 with both the elastic and clipping functions as well

### BRIEF DESCRIPTION OF THE DRAWINGS

FIG. 1 is a schematic representation of the known method used for doing exercise of opening mouth thereby;

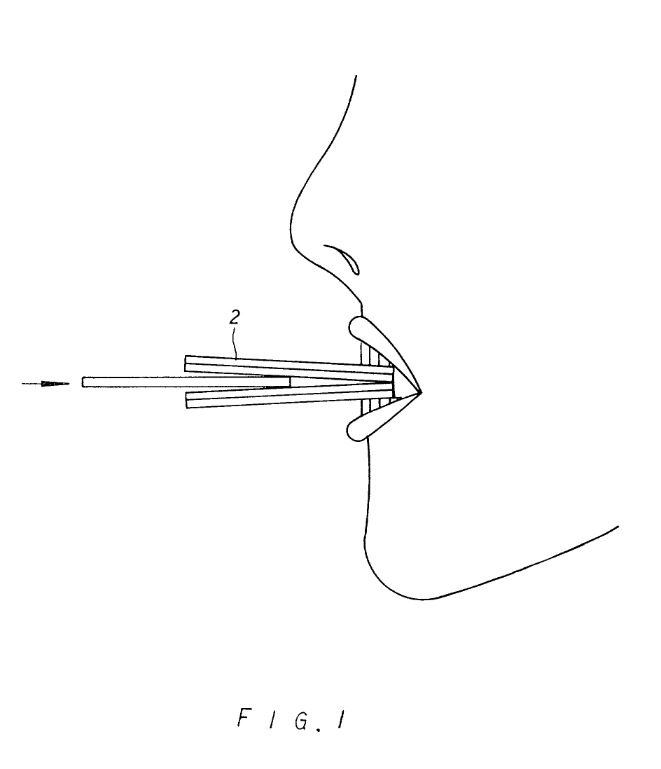

FIG. 2 is a schematic exploded perspective view of an expender provided by the present invention;

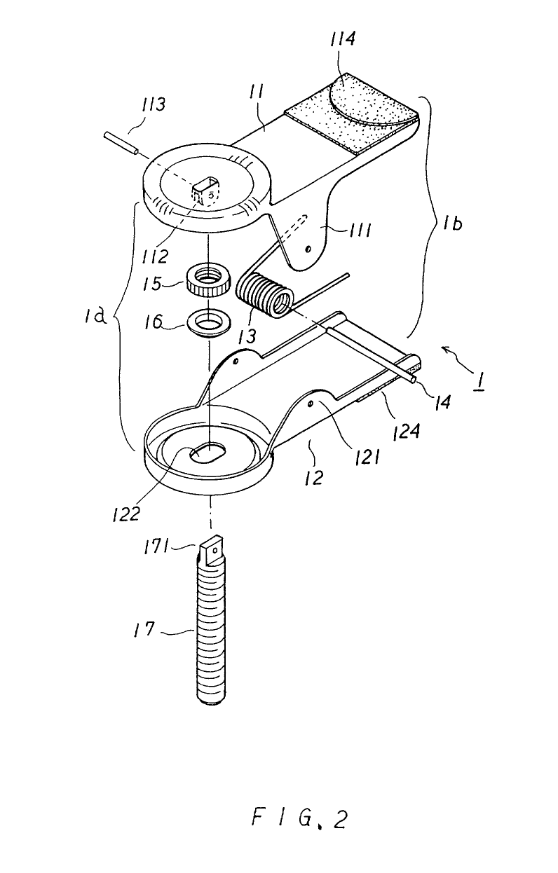

FIG. 3 is the first schematic representation of the present invention illustrating the exercising and operating method used thereby;

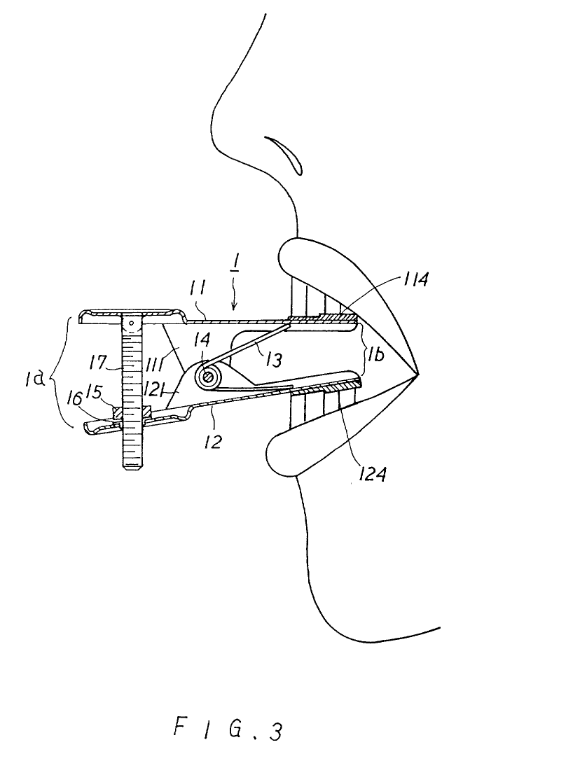

FIG. 4 is the second schematic representation of the present invention illustrating the exercising and operating method used thereby;

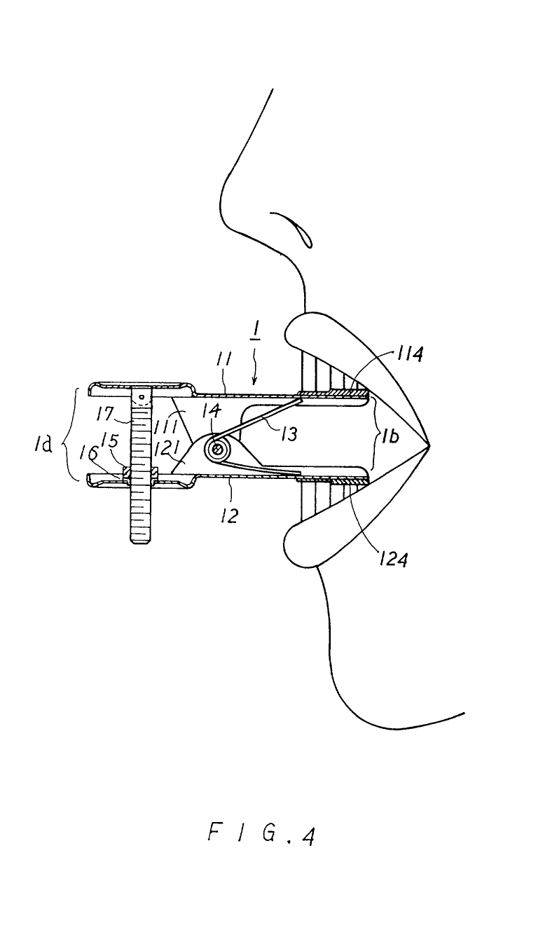

FIG. 5 is a schematic exploded perspective view of an expender provided by another embodiment in accordance with the present invention;

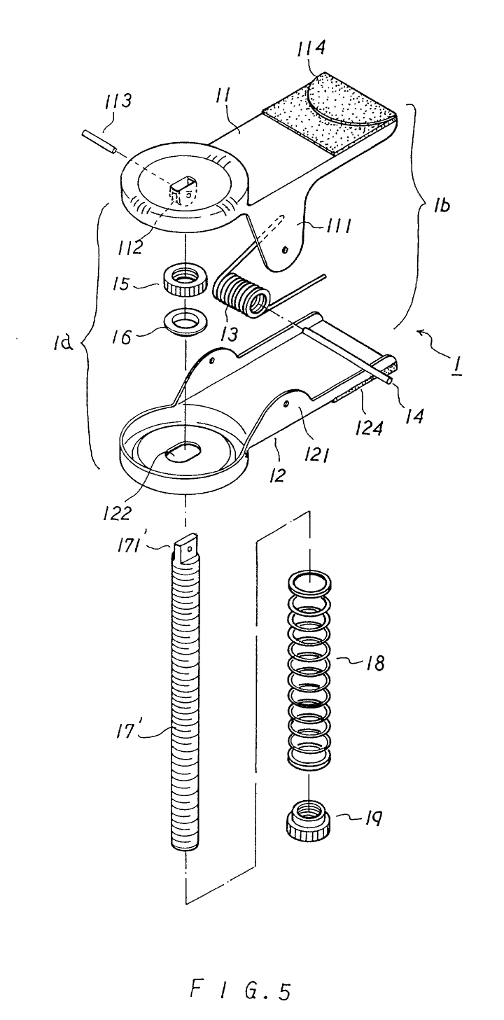

FIG. 6 is a side cross-sectional view of an expender provided by another embodiment i n accordance with the present invention;

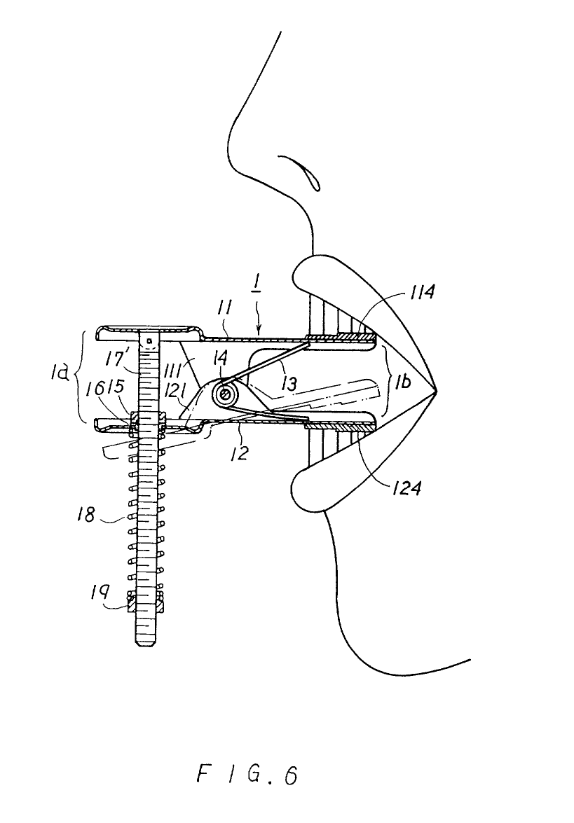

FIG. 7 is a schematic view of an elastic clip provided by another embodiment in accordance with the present invention.

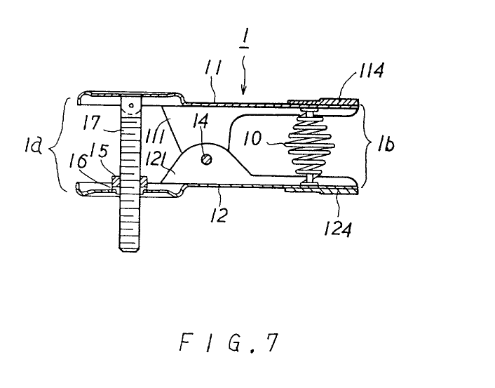

What is claimed is:

1. An expander for the oral cavity's rehabilitation, comprising an elastic clip consisting of an upper brace and a lower brace, which are pivoted together and with a function of elastic clipping, wherein the said elastic clip comprising an adjusting end and a occlusive end, and also there is a screw, which is pushed through a long boring pierced into the center portion of a lower brace after it has been screwed on with a nut thereat, is pivoted onto the upper brace positioned at the said adjusting end, and in this way the said occlusive end bitten by both the upper and lower gums of a user would be able to open slowly, while screwing the nut screwed onto the said screw pivoted onto the said adjusting end, and thus having a patient to open her or his mouth further, and in that way it would be applicable to proceed the tense training program repeatedly on the occlusive musculature consequently.

2. An expander for the oral cavity's rehabilitation of claim 1, wherein a spring is mounted at the portion in-between both the upper and lower braces, which are pivoted together, of the said elastic clip, and thus the said adjusting end of the said elastic clip would be under a constant-opening condition while in the mean time the said occlusive end would then under a constant-closing condition instead.

3. An expander for the oral cavity's rehabilitation of claim 1, wherein the said screw would be mounted with a spring and also screwed on a nut successively after it had been pushed through the said lower brace therefrom.

4. An expander for the oral cavity's rehabilitation of claim 1, wherein a soft padding is fixed onto the occlusal plane of both the upper/lower braces of the said occlusive end.

5. An expander for the oral cavity's rehabilitation of claim 1, wherein the spring mounted at the portion in-between the upper/lower braces of the said elastic clip therefrom could be either a compression spring or a torque helical spring.
